(19) United States
(12) Patent Application Publication
(10) Pub. No.: US 2024/0290430 A1
(43) Pub. Date: Aug. 29, 2024

(54) COMPUTER IMPLEMENTED METHOD AND SEQUENCE DESIGNING SYSTEM FOR DESIGNING CELL NUCLEOTIDE SEQUENCES

(71) Applicant: Wipro Limited, Bangalore (IN)

(72) Inventors: Manoj MADHUSUDHANAN, Bangalore (IN); Sreekumar CHOYARMADATHIL, Bangalore (IN); Parul BERRY, Jalandhar (IN)

(21) Appl. No.: 18/298,640

(22) Filed: Apr. 11, 2023

(30) Foreign Application Priority Data
Feb. 24, 2023 (IN) 202341012638

(51) Int. Cl. G16B 30/20; G06N 5/022

(52) U.S. Cl. CPC G16B 30/20; G06N 5/022

(57) ABSTRACT

The present disclosure relates to field of cell nucleotide sequence designing and discloses method and system for designing cell nucleotide sequences. The sequence designing system receives historical data related to results of procedures related to analysis of cell nucleotide sequences from databases. Further, the sequence designing system executes an Artificial Intelligence (AI) based prediction model using vectorized data corresponding to the historical data. Thereafter, the sequence designing system predicts a plurality of cell nucleotide sequences having values of cell characteristics within a predefined threshold values of the cell characteristics for a target cell nucleotide sequence using the AI based prediction model. Furthermore, the sequence designing system identifies feasible cell nucleotide sequences among the plurality of cell nucleotide sequences based on predefined reference information. Finally, the sequence designing system generates an explanation for the ranked list of the feasible cell nucleotide sequences, thereby designing the cell nucleotide sequences.

### TECHNICAL FIELD

The present disclosure, in general, relates to Artificial Intelligence (AI) based designing of cell nucleotide sequences, and particularly relates to a computer implemented method and sequence designing system for designing cell nucleotide sequences.

### BACKGROUND

Cell therapy and gene therapy are emerging as effective methods for multiple complications. The applications of this include immunotherapy for oncology, regenerative medicine, and gene therapy for hemoglobinopathies. All these methods have applicability in both, rare and common diseases. In all these therapeutic areas, for specific applications, the nucleotide sequence of the cell is edited after designing the best nucleotide sequence. The existing processes for identifying the best cell nucleotide sequence are based on in-vivo and experimental approach, where finding the most efficient edits is time-consuming and not scalable due to the human and lab involvement at each step.

For example, one of the existing methods, called Chimeric Antigen Receptor T-cell (CAR T-cell) therapy, utilizes human immune system to mount a targeted response against cancer cells. The CAR T-cells consist of an extracellular target binding domain attached to the intracellular signaling domain through the hinge and transmembrane anchoring protein. They have become a part of modern medicine and are used to treat aggressive B-cell lymphomas. Further, the T-cells obtained from a patient are re-engineered in a laboratory to produce CAR and CAR T-cells are then infused back into the patient. However, the major criticism of CAR T-cell therapy is that it's very expensive. Currently, CAR T-cells are designed separately for each individual patient resulting in huge cost for each treatment.

Further, identification of novel targets appropriate for CAR T-cell therapy is the main determinant in the expansion of applications of CAR T-cell therapy. Currently, CAR T-cell therapies targeting B-lymphocyte antigen (CD19) and B-cell Maturation Antigen (BCMA) have only been approved. However, several CAR T-cell therapies targeting many other antigens are in development with many ongoing clinical trials. Tumor heterogeneity, identification of novel targets on solid tumor cells, and overcoming physical barriers in the tumor microenvironment are major obstacles in deploying CAR T-cell therapies. The current process of identifying the most efficient edit (cell nucleotide sequence) for CAR T-cell therapies is very time-consuming and lacks scalability due to the involvement of humans and laboratory resources at each step. Multiple preclinical studies and clinical trials of CAR T-cell therapies targeting different antigens in a tumor are conducted without any optimization in target cell nucleotide sequence selection. The lack of a standardized method of narrowing down target cell nucleotide sequences leads to higher failure rates in these studies with a loss of time and resources. The inefficacy of this approach slows down the expansion of CAR T-cell therapies to various solid tumors. Hence, the existing approaches do not address the problem related to predicting of the optimum nucleotide sequences required for the modified cell in cell therapy and gene therapy for a specific disease antigen.

The information disclosed in this background of the disclosure section is only for enhancement of understanding of the general background of the invention and should not be taken as an acknowledgement or any form of suggestion that this information forms the prior art already known to a person skilled in the art.

### SUMMARY

Disclosed herein is a computer implemented method for designing cell nucleotide sequences. The method comprises receiving, by a sequence designing system, historical data related to results of one or more procedures related to analysis of cell nucleotide sequences from one or more databases. Further, the method comprises executing an Artificial Intelligence (AI) based prediction model using vectorized data corresponding to the historical data. Thereafter, the method comprises predicting a plurality of cell nucleotide sequences having values of one or more cell characteristics within a predefined threshold values of the one or more cell characteristics for a target cell nucleotide sequence using the AI based prediction model. Furthermore, the method comprises identifying one or more feasible cell nucleotide sequences among the plurality of cell nucleotide sequences based on predefined reference information. The one or more feasible cell nucleotide sequences is each assigned with a rank to generate a ranked list. Finally, the method comprises generating an explanation for the ranked list of the one or more feasible cell nucleotide sequences, thereby designing the cell nucleotide sequences.

Further, the present disclosure relates to a sequence designing system for designing cell nucleotide sequences. The sequence designing system comprises a processor and a memory. The memory is communicatively coupled to the processor and stores processor-executable instructions, which on execution, cause the processor to receive historical data related to results of one or more procedures related to analysis of cell nucleotide sequences from one or more databases. Further, the instructions cause the processor to execute an Artificial Intelligence (AI) based prediction model using vectorized data corresponding to the historical data. Thereafter, the instructions cause the processor to predict a plurality of cell nucleotide sequences having values of one or more cell characteristics within a predefined threshold values of the one or more cell characteristics for a target cell nucleotide sequence using the AI based prediction model. Furthermore, the instructions cause the processor to identify one or more feasible cell nucleotide sequences among the plurality of cell nucleotide sequences based on predefined reference information. The one or more feasible cell nucleotide sequences is each assigned with a rank to generate a ranked list. Finally, the instructions cause the processor to generate an explanation for the ranked list of the one or more feasible cell nucleotide sequences, thereby designing the cell nucleotide sequences.

Furthermore, the present disclosure relates to a non-transitory computer readable medium including instructions stored thereon that when processed by at least one processor, cause a sequence designing system to perform operations comprising receiving historical data related to results of one or more procedures related to analysis of cell nucleotide sequences from one or more databases. Further, the instructions cause the processor to execute an Artificial Intelligence (AI) based prediction model using vectorized data corresponding to the historical data. Thereafter, the instructions cause the processor to predict a plurality of cell nucleotide sequences having values of one or more cell characteristics within a predefined threshold values of the one or more cell characteristics for a target cell nucleotide sequence using the AI based prediction model. Furthermore, the instructions cause the processor to identify one or more feasible cell nucleotide sequences among the plurality of cell nucleotide sequences based on predefined reference information. The one or more feasible cell nucleotide sequences is each assigned with a rank to generate a ranked list. Finally, the instructions cause the processor to generate an explanation for the ranked list of the one or more feasible cell nucleotide sequences, thereby designing the cell nucleotide sequences.

The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTION OF THE ACCOMPANYING DRAWINGS

The accompanying drawings, which are incorporated in and constitute a part of this disclosure, illustrate exemplary embodiments and, together with the description, explain the disclosed principles. In the figures, the left-most digit(s) of a reference number identifies the figure in which the reference number first appears. The same numbers are used throughout the figures to reference like features and components. Some embodiments of system and/or methods in accordance with embodiments of the present subject matter are now described, by way of example only, and regarding the accompanying figures, in which:

FIG. 1 shows an overview of functioning of a sequence designing system in accordance with some embodiments of the present disclosure.

FIG. 2 shows a detailed block diagram of a sequence designing system in accordance with some embodiments of the present disclosure.

FIG. 3A shows a flowchart illustrating a method for designing cell nucleotide sequences, in accordance with some embodiments of the present disclosure.

FIG. 3B shows a flowchart illustrating a method for identifying one or more feasible cell nucleotide sequences, in accordance with some embodiments of the present disclosure.

FIG. 4 shows an exemplary architecture of the proposed sequence designing system, in accordance with some embodiments of the present disclosure.

FIG. 5A shows an exemplary illustration of a predicted T-cell sequence, in accordance with some embodiments of the present disclosure.

FIG. 5B shows an exemplary illustration of a predicted T-cell sequence with explanation and values, in accordance with some embodiments of the present disclosure.

FIG. 6 illustrates an exemplary computer system communicating with one or more databases via communication network, for designing cell nucleotide sequence.

It should be appreciated by those skilled in the art that any block diagrams herein represent conceptual views of illustrative systems embodying the principles of the present subject matter. Similarly, it will be appreciated that any flow charts, flow diagrams, state transition diagrams, pseudo code, and the like represent various processes which may be substantially represented in computer readable medium and executed by a computer or processor, whether such computer or processor is explicitly shown.

### DETAILED DESCRIPTION

In the present document, the word “exemplary” is used herein to mean “serving as an example, instance, or illustration.” Any embodiment or implementation of the present subject matter described herein as “exemplary” is not necessarily to be construed as preferred or advantageous over other embodiments.

While the disclosure is susceptible to various modifications and alternative forms, specific embodiment thereof has been shown by way of example in the drawings and will be described in detail below. It should be understood, however that it is not intended to limit the disclosure to the specific forms disclosed, but on the contrary, the disclosure is to cover all modifications, equivalents, and alternative falling within the scope of the disclosure.

The terms “comprises”, “comprising”, “includes”, or any other variations thereof, are intended to cover a non-exclusive inclusion, such that a setup, device, or method that comprises a list of components or steps does not include only those components or steps but may include other components or steps not expressly listed or inherent to such setup or device or method. In other words, one or more elements in a system or apparatus proceeded by “comprises . . . a” does not, without more constraints, preclude the existence of other elements or additional elements in the system or method.

In an embodiment, the present disclosure proposes an Artificial Intelligence (AI) based approach to design immune evasive cell nucleotide sequences that demonstrate cell fitness with respect to Endoplasmic Reticulum (ER) stress and cytokine stress and display substantial binding capacity against the target antigens. Through the proposed invention, the aim is to filter a large number of possible cell nucleotide sequences to a few hundred predicted cell nucleotide sequences. Any proven methods like Clustered Regularly Interspaced Short Palindromic Repeats (CRISPR) may be used to do further in-vitro testing of the top predicted cell nucleotide sequences by the proposed system. Further, to decrease the side-effect profile of cell therapy, such as Chimeric Antigen Receptor T-cell (CAR T-cell) therapy, the proposed invention suggests modifications to the cell nucleotide sequences to prevent adverse host immune response to increase immune evasiveness of engineered cells. These sequence modifications are decided based on the epitopes of the antigen, as in many cases the antigen has multiple mutations. The proposed invention further suggests gene editing to optimize cell fitness in face of cytokine stress, ER stress, and rejection by natural killer cells. Through the proposed AI based prediction model, the one or more feasible cell nucleotide sequences for targeting a particular antigen sequence are predicted. The invention also proposes to rank one or more cell nucleotide sequences and provides explanation for the selection of one or more feasible cell nucleotide sequences.

In an embodiment, the proposed method aims to identify one or more feasible cell nucleotide sequence among a large number of possible nucleotide sequences. This helps in designing successful cell nucleotide sequences at a faster rate with high efficiency as the number of possible sequences is reduced.

In the following detailed description of the embodiments of the disclosure, reference is made to the accompanying drawings that form a part hereof, and in which are shown by way of illustration specific embodiments in which the disclosure may be practiced. These embodiments are described in sufficient detail to enable those skilled in the art to practice the disclosure, and it is to be understood that other embodiments may be utilized and that changes may be made without departing from the scope of the present disclosure. The following description is, therefore, not to be taken in a limiting sense.

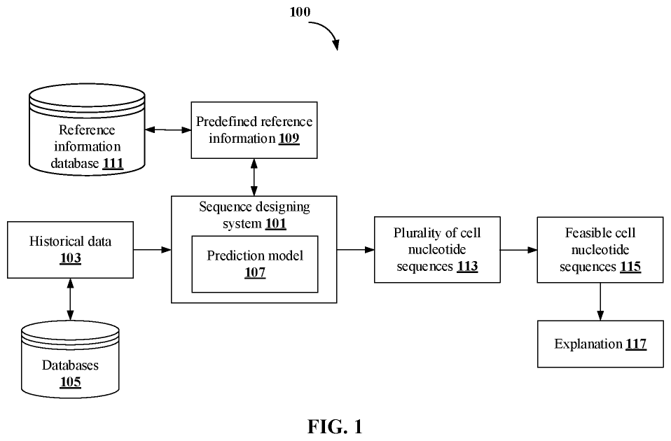

FIG. 1 shows an overview of functioning of a sequence designing system, in accordance with some embodiments of the present disclosure.

In an embodiment, a sequence designing system 101, may be configured and used for designing cell nucleotide sequences. The sequence designing system 101 may be any computing unit that may be configured to design cell nucleotide sequences. As an example, the computing device may include, without limiting to, a desktop computer, a laptop and the like.

In an embodiment, the sequence designing system 101 may be configured to receive historical data 103 related to results of one or more procedures related to analysis of cell nucleotide sequences from one or more databases 105. The historical data 103 may include, without limitation, at least one of one or more antigen nucleotide sequences, one or more cell nucleotide sequences, biomarkers data, function-specific biomarkers data, clinical trial progression data, clinical trial outcome data, in-vitro progression data, and in-vitro outcome data. The one or more procedures may include, without limitation, clinical trials, in-vitro research, and approved immunotherapy. The results of the one or more procedures may be stored in the one or more databases 105. The one or more databases 105 may include, without limitation, an external database and internal database. The external database may be publicly available database of the results of the one or more procedures. The internal database may be a database available within an organization and data available from regulatory agencies. The organization may be the organization designing the cell nucleotide sequences. As an example, the external database and the internal database may be accessed by the sequence designing system 101 over an intranet connection. The external database and the internal database may also be connected physically with the sequence designing system 101.

Upon receiving the historical data 103, the sequence designing system 101 may perform one or more pre-processing operations on the historical data 103 for verifying correctness and completeness of the historical data 103 based on predefined reference information 109 stored in reference information database 111. As an example, the reference information database 111 may also be accessed by the sequence designing system 101 over an intranet connection. Alternatively, the reference information database 111 may be connected physically with the sequence designing system 101. The predefined reference information 109 may include, without limitation, at least one of a three-dimensional (3D) structure and binding database, a gene regulatory and epigenetic database, a target selectivity database, a signaling pathways database, an inflammatory and Endoplasmic Reticulum (ER) stress database, and a threshold range database. In other words, the predefined reference information 109 may include scientific data from databases such as, without limitation, PubMed, National Institutes of Health (NIH), National Health Service (NHS), AlphaFold and the like. As part of the pre-processing operation, the sequence designing system 101 may send one or more queries to the reference information database 111 to verify correctness and completeness of the historical data 103. The sequence designing system 101 may retain the correct and complete historical data 103 for further processing and discard the incorrect and incomplete historical data 103. Further, the sequence designing system 101 may arrange the historical data 103 in a chronological order based on timestamps associated with the historical data 103. Upon arranging the historical data 103 in the chronological order, the sequence designing system 101 may vectorize the historical data 103 using known vectorizing techniques. As an example, the vectorizing techniques may include, any of the vector embedding for nucleotide sequence and biomarkers without limitation, Word2Vec and the like.

In an embodiment, upon verifying the correctness and the completeness of the historical data 103, the sequence designing system 101 executes an Artificial Intelligence (AI) based prediction model 107 (also referred as prediction model 107) using vectorized data corresponding to the historical data 103. The sequence designing system 101 may align the vectorized representation of the data in time-series, representing the trajectory of the historical data 103. The sequence designing system 101 may utilize transformer-based self-learning mechanism that learns by predicting the masked data in the training data set i.e., time-series aligned vectorized input data and uses a gradient descent to minimize the prediction error. Further, the sequence designing system 101 may use transformer and Graph Neural Network (GNN) for representing the learned knowledge enabling representation at local and global level. The sequence designing system 101 executes a learned joint representation/model (multi-dimensional mathematical space) of the time-series data. As an example, the AI based prediction model 107 may represent the clusters for all recovering patient cases together, worsening patient cases together and no effect patient cases together. Further, the AI based prediction model 107 may show, for a given antigen nucleotide sequence, what cell nucleotide sequences worked, what cell nucleotide sequences did not work and what cell nucleotide sequences did not have any effect on the patient. The AI based prediction model 107 may also include the corresponding historical data 103. When new historical data 103 is available, the sequence designing system 101 may incrementally train and update the AI based prediction model 107.

In an embodiment, upon executing the AI based prediction model 107, the sequence designing system 101 predicts a plurality of cell nucleotide sequences 113 having values of one or more cell characteristics within a predefined threshold values of the one or more cell characteristics for a target cell nucleotide sequence using the AI based prediction model 107. The sequence designing system 101 may determine an equilibrium dissociation constant (K_{D}) related to binding affinity, value of biomarkers related to at least one of immune evasion and cell fitness using the AI based prediction model 107. The sequence designing system 101 may receive a target antigen nucleotide sequence from a doctor, a physician, or a scientist of a patient for whom the cell nucleotide sequences need to be determined. Alternatively, the target antigen nucleotide sequence may be taken automatically from a database of patient information. Alternatively, the the target antigen nucleotide sequence can be for a group of patients with similar target antigen nucleotide sequence for identification of appropriate cell nucleotide sequences. The sequence designing system 101 may receive the target cell nucleotide sequence from a user using an user interface associated with the sequence designing system 101. The user may include, without limitation, at least one of the doctors, the physician, and the scientist. The sequence designing system 101 may utilize transformer-based self-learning algorithms to predict the plurality of cell nucleotide sequences 113 for the target antigen nucleotide sequence of the patient. Also, the sequence designing system 101 may decode the vectors in the regions of success in the AI based prediction model 107 for the best cell nucleotide sequences in terms of the one or more cell characteristics individually corresponding to the target cell nucleotide sequence.

In an embodiment, upon predicting the plurality of cell nucleotide sequences 113, the sequence designing system 101 identifies one or more feasible cell nucleotide sequences 115 among the plurality of cell nucleotide sequences 113 based on the predefined reference information 109. The one or more feasible cell nucleotide sequences 115 is each assigned with a rank to generate a ranked list. The sequence designing system 101 may extract a feasibility data corresponding to feasibility of each of the plurality of cell nucleotide sequences 113 from the predefined reference information 109, based on the equilibrium dissociation constant (K_{D}) and value of biomarkers related to at least one of immune evasion and cell fitness. As an example, the sequence designing system 101 may send one or more queries to the reference information database 111 that is storing the predefined reference information 109. Consider an exemplary verification scenario for verifying the plurality of cell nucleotide sequences 113. the reference information database 111 stores one or more databases related to the scientific data. To verify one or more feasible cell nucleotide sequences for binding affinity, intermediate 3D structure level prediction corresponding to the cell nucleotide sequence modifications and binding affinity data (K_{D}) to the identified target is required, which is available in the 3D Structure and Binding database, the gene regulatory and the epigenetic database and the target selectivity database which is stored in the reference information database 111. Similarly, to verify one or more feasible cell nucleotide sequences for immune evasion, value of biomarkers related to immune evasion is required which is available in the signaling pathways database which is stored in the reference information database 111. Further, to verify one or more feasible cell nucleotide sequences for cell fitness, value of biomarkers related to cell fitness is required which is available in the inflammatory and Endoplasmic Reticulum (ER) stress database which is stored in the reference information database 111. The sequence designing system 101 may retain the verified one or more feasible cell nucleotide sequences 115 among the plurality of cell nucleotide sequences 113 and discard the unfeasible cell nucleotide sequences among the plurality of cell nucleotide sequences 113.

Upon extracting the feasibility data corresponding to feasibility of each of the plurality of cell nucleotide sequences 113, the sequence designing system 101 may select one or more of the plurality of cell nucleotide sequences 113 within predefined threshold ranges of at least one of immune evasion and cell fitness. The predefined threshold ranges may be threshold ranges for a human body. The threshold ranges may be available in threshold range database present in the reference information database 111. The sequence designing system 101 may receive positive or negative result from the reference information database 111. The positive result may indicate that the immune evasion and cell fitness is within the predefined threshold ranges, subsequent to which the sequence designing system 101 may retain the one or more feasible cell nucleotide sequences 115. The negative result may indicate that the immune evasion and cell fitness is not within the predefined threshold ranges and the sequence designing system 101 may discard the one or more feasible cell nucleotide sequences 115. Thereafter, the sequence designing system 101 may generate a ranked list of the one or more feasible cell nucleotide sequences 115 based on K_{D}. The one or more feasible cell nucleotide sequences 115 with lowest K_{D} value have the highest binding affinity and is given the highest ranking in the ranked list. The ranked list may be arranged as per highest to lowest ranking of the binding affinity.

In an embodiment, upon identifying the one or more feasible cell nucleotide sequences 115, the sequence designing system 101 generates an explanation 117 for the ranked list of the one or more feasible cell nucleotide sequences 115, thereby designing the cell nucleotide sequences. The explanation 117 may be generated by utilizing at least one of inverse folding, concept activation vectors and causality techniques. The inverse folding techniques may be used to explain the target binding capability of the one or more feasible cell nucleotide sequences 115. The concept activation vectors may be used to explain immune evasion and cell fitness of the one or more feasible cell nucleotide sequences 115. The causality methods are used to explain the action mechanism, including pathways of the one or more feasible cell nucleotide sequences 115. The explanation 117 may include binding affinity values considered for the one or more cell nucleotide sequences for binding and values of biomarkers considered for the one or more cell nucleotide sequences for immune evasion and cell fitness. The explanation 117 may be presented to a user. The user may include, without limitation, at least one of the doctors, the physician, and the scientist. As an example, the explanation 117 may be presented on a display device associated with the sequence designing system 101. The explanation 117 may be sent to the user via an email or any other data sharing service.

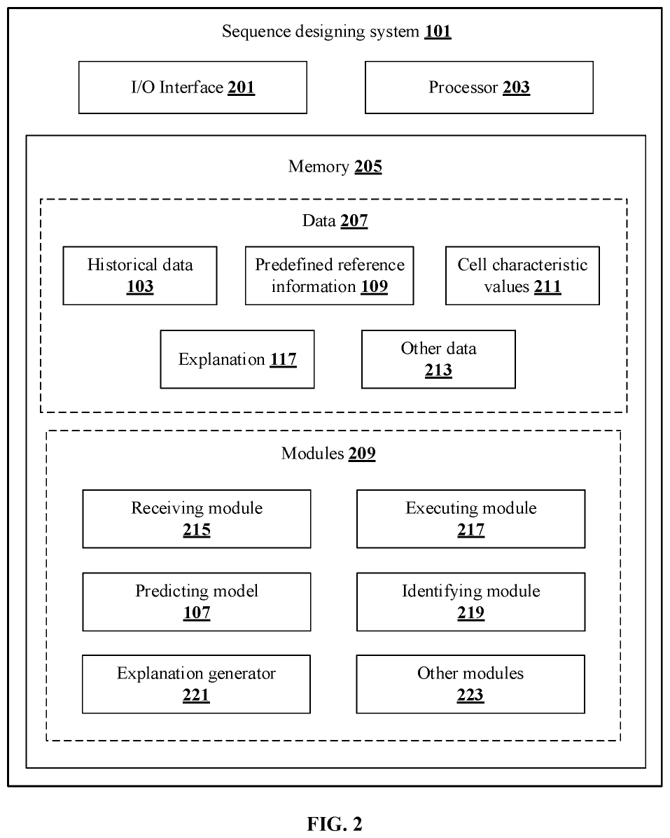

FIG. 2 shows a detailed block diagram of a sequence designing system 101 in accordance with some embodiments of the present disclosure.

In an embodiment, the sequence designing system 101 may include an I/O interface 201, a processor 203 and a memory 205. The processor 203 may be configured to perform one or more functions of the sequence designing system 101 for designing cell nucleotide sequences, using the data 207 and the one or more modules 209 stored in a memory 205 of the sequence designing system 101. In an embodiment, the memory 205 may store data 207 and one or more modules 209.

In an embodiment, the data 207 may be stored in the memory 205 may include, without limitation, historical data 103, predefined reference information 109, one or more cell characteristic values 211, an explanation 117 and other data 213. In some implementations, the data 207 may be stored within the memory 205 in the form of various data structures. Additionally, the data 207 may be organized using data models, such as relational or hierarchical data models. The other data 213 may include various temporary data and files generated by the one or more modules 209.

In an embodiment, the historical data 103 is data related results of one or more procedures related to analysis of cell nucleotide sequences stored in one or more databases 105. The one or more procedures may include, without limitation, clinical trials, in-vitro research and approved immunotherapy. The results of one or more procedures may be stored in the one or more databases 105. The one or more databases 105 may include, without limitation, an external database and internal database. The external database may be publicly available database of the results of the one or more procedures. The internal database may be database available within an organization and data available from regulatory agencies. The historical data 103 may include, without limitation, at least one of one or more antigen nucleotide sequences, one or more cell nucleotide sequences, biomarkers data, function-specific biomarkers data, clinical trial progression data, clinical trial outcome data, in-vitro progression data, and in-vitro outcome data. The one or more antigen nucleotide sequences and one or more cell nucleotide sequences may be the nucleotide sequences of the antigens such as cancer and the Thymus cell (T-cell) such as a Chimeric Antigen Receptor (CAR) T-cell utilized in one or more procedures. The biomarkers data may be numerical values of different biomarkers such as an Interleukin-6 (IL-6) value, White Blood Cells (WBC) and the like. The biomarkers data may be recorded during the one or more procedures. The function-specific biomarkers data may be numerical values of biomarkers related to specific functions such as binding affinity, immune evasion and cell fitness recorded in one or more procedures. As an example, the binding affinity is captured by parameters such as an equilibrium dissociation constant (K_{D}), immune evasion by immunity related biomarkers such as IL-6 value and cell fitness by biomarkers that measure the cell related biomarkers such as T-cell count and cell exhaustion. The clinical trial progression data may indicate progression of the clinical trial. The clinical trial outcome data may indicate success or failure of the clinical trial. The in-vitro progression data may indicate progression of the in-vitro research. The in-vitro outcome data may indicate success or failure of the in-vitro research. In an embodiment, one or more pre-processing operations may be performed on the historical data 103 for verifying correctness and completeness of the historical data 103 based on the predefined reference information 109. Further, the historical data 103 may be arranged in a chronological order based on a timestamps s associated with the historical data 103. Thereafter, the historical data 103 may be vectorized using any of the vector embedding for the cell nucleotide sequences and biomarkers without limitation, Word2Vec and the like. The vectorized data corresponding to the historical data 103 may be used to execute an Artificial Intelligence (AI) based prediction model 107.

In an embodiment, the predefined reference information 109 is information stored in reference information 109 related to scientific information from databases such as PubMed, National Institutes of Health (NIH), National Health Service (NHS), AlphaFold and the like. The predefined reference information 109 may include, without limitation, at least one of a three-dimensional (3D) structure and binding database, a gene regulatory and epigenetic database, a target selectivity database, a signaling pathways database, an inflammatory and Endoplasmic Reticulum (ER) stress database, and a threshold range database. The 3D structure and binding database stores the 3D structure of the antigen and cell nucleotide sequences. The gene regulatory and epigenetic database stores the non-coding part of the antigen and cell nucleotide sequences and the human epigenetic factors. The target selectivity database stores the information on target selectivity, to ensure the necessity of only targeting the antigens with no impact to other cells. The signaling pathways database stores the known cell signaling pathways and the biochemical pathways. The inflammatory and Endoplasmic Reticulum (ER) stress database stores the information related to inflammatory stress and ER stress that includes an Unfolded Protein Response (UPR). The threshold range database stores the range of all the biomarkers of one or more cell characteristics for the normal human cases. In an embodiment, the predefined reference information 109 is used to verify correctness and completeness of the historical data 103. Further, the predefined reference information 109 is used to identify one or more feasible cell nucleotide sequences 115 among plurality of cell nucleotide sequences 113 predicted by the AI based prediction model 107.

In an embodiment, the one or more cell characteristic values 211 is values related to the one or more cell characteristics. The one or more cell characteristics may include, without limitation, binding affinity, immune evasion, and cell fitness. The binding affinity of the cell nucleotide sequence is the strength of binding interaction between the cell nucleotide sequence and a target cell nucleotide sequence. As an example, the equilibrium dissociation constant (K_{D}) may be used to determine the binding affinity. The predicted cell nucleotide sequences with lowest K_{D} value have the highest binding affinity. The immune evasion is a process in which the predicted cell nucleotide sequences can bypass the immune system of a human and continue growth and transmission of the predicted cell nucleotide sequence in the human body. As an example, immunity related biomarkers such as IL-6 value may be used to determine the immune evasion. The cell fitness is an ability of the predicted cell nucleotide sequences to thrive in a human body. As an example, T-cell count, and cell exhaustion may be used to determine the cell fitness. The one or more cell characteristic values of the predicted cell nucleotide sequences may be within predefined threshold values of the one or more cell characteristics 211 for the target cell nucleotide sequence.

In an embodiment, the explanation 117 is an information related to one or more feasible cell nucleotide sequences 115 based on the one or more cell characteristics. The explanation 117 may include values of the one or more cell characteristics 211 related to the one or more feasible nucleotide sequences.

In an embodiment, the data 207 may be processed by the one or more modules 209 of the sequence designing system 101. In some implementations, the one or more modules 209 may be communicatively coupled to the processor 203 for performing one or more functions of the sequence designing system 101. In an implementation, the one or more modules 209 may include, without limiting to, a receiving module 215, a executing module 217, a predicting model 107, an identifying module 219, an explanation generator 221 and other modules 223.

As used herein, the term module may refer to an Application Specific Integrated Circuit (ASIC), an electronic circuit, a hardware processor (shared, dedicated, or group) and memory that execute one or more software or firmware programs, a combinational logic circuit, and/or other suitable components that provide the described functionality. In an implementation, each of the one or more modules 209 may be configured as stand-alone hardware computing units. In an embodiment, the other modules 223 may be used to perform various miscellaneous functionalities of the sequence designing system 101. It will be appreciated that such one or more modules 209 may be represented as a single module or a combination of different modules.

In an embodiment, the receiving module 215 may be configured for receiving historical data 103 related to results of one or more procedures related to analysis of cell nucleotide sequences from one or more databases 105. The receiving module 215 may perform one or more pre-processing operations on the historical data 103 for verifying correctness and completeness of the historical data 103 based on the predefined reference information 109. The receiving module 215 may check the historical data 103 with the predefined reference information 109 present in the reference information database 111. As an example, one or more queries may be sent to the reference information database 111 to verify correctness and completeness of the historical data 103. Consider two exemplary scenarios mentioned below, which are used to verify the historical data 103:

### Example 1

Query 1: Is data related to clinical trial 1 correct?
Biomarker: IL-6 value is 60 picograms per milliliter (pg/ml) (high)
Clinical trial/in-vitro outcome/progression: Patient died.
Result 1: “Yes”, input data match with data pre-stored in reference information database
Explanation 1: Biomarker indicator from clinical trial is aligned with outcome of the clinical trial when compared with known scientific information.

### Example 2

Query 2: Is data related to clinical trial 1 correct?
Biomarker: IL-6 value is 60 pg/ml (high)
Clinical trial/in-vitro outcome/progression: Patient recovered.
Result 2: “No”, Input data mismatch with data pre-stored in reference information database
Explanation 2: Biomarker indicator from clinical trial is not aligned with outcome of the clinical trial when compared with known scientific information.

As shown in the above exemplary scenarios, the historical data 103 present in the example 1 is correct and complete, therefore the historical data 103 can be used for further operations. However, in example 2, the historical data 103 present is incomplete, therefore the historical data 103 is discarded and not used for further operations. The receiving module 215 may retain the correct and complete historical data 103 for further operations and discard the incorrect and incomplete historical data 103. Further, the receiving module 215 may arrange the historical data 103 in a chronological order based on timestamps associated with the historical data 103. Thereafter, the receiving module 215 may vectorize the historical data 103 to be used to execute an Artificial Intelligence (AI) based prediction model 107. The historical data 103 may be vectorized using any of the vector embedding for cell nucleotide sequences and biomarkers without limitation, Word2Vec and the like.

In an embodiment, the executing module 217 may be configured for executing an Artificial Intelligence (AI) based prediction model 107 using vectorized data corresponding to the historical data 103. The executing module 217 may utilize transformer-based self-learning mechanism that learns by predicting the masked data in the training data set i.e., time-series aligned vectorized input data and uses a gradient descent to minimize the prediction error. Further, the executing module 217 may use transformer and Graph Neural Network (GNN) for representing the learned knowledge enabling representation at local and global level. This unit creates a learned joint representation/model (multi-dimensional mathematical space) of the time-series data. As an example, the AI based prediction model 107 may represent the clusters for all the recovering patient cases together, worsening patient cases together and no effect patient cases together. Further, the AI based prediction model 107 may show, for a given antigen nucleotide sequence, what cell nucleotide sequences worked, what cell nucleotide sequences did not work and what cell nucleotide sequences did not have any effect on the patient. The AI based prediction model 107 may also include the corresponding historical data 103.

In an embodiment, the prediction model 107 (also referred as AI based prediction model 107) may be a pre-trained model configured for predicting a plurality of cell nucleotide sequences 113 having values of one or more cell characteristics 211 within a predefined threshold values of the one or more cell characteristics 211 for a target cell nucleotide sequence using the AI based prediction model 107. Further, the prediction model 107 may determine an equilibrium dissociation constant (K_{D}) related to binding affinity, value of biomarkers related to at least one of immune evasion and cell fitness.

In an embodiment, the identifying module 219 may be configured for identifying one or more feasible cell nucleotide sequences 115 among the plurality of cell nucleotide sequences 113 based on predefined reference information 109. The one or more feasible cell nucleotide sequences 115 is each assigned with a rank to generate a ranked list. The identifying module 219 may extract a feasibility data corresponding to feasibility of each of the plurality of cell nucleotide sequences 113 from the predefined reference information 109, based on an equilibrium dissociation constant (K_{D}) and value of biomarkers related to at least one of immune evasion and cell fitness. As an example, the identifying module 219 may send one or more queries to the reference information database 111 storing the predefined reference information 109. The predefined reference information 109 may include, without limitation, at least one of a three-dimensional (3D) structure and binding database, a gene regulatory and epigenetic database, a target selectivity database, a signaling pathways database, an inflammatory and Endoplasmic Reticulum (ER) stress database, and a threshold range database. Consider exemplary verification scenarios for verifying the plurality of cell nucleotide sequences 113. For verification of cell nucleotide sequences for binding affinity intermediate 3D structure level prediction corresponding to the cell nucleotide sequence modifications and binding affinity data (K_{D}) to the identified target is required which is available in the 3D Structure and Binding Database, the gene regulatory and the epigenetic database and the target selectivity database. For verification of cell nucleotide sequences for immune evasion, value of biomarkers related to immune evasion is required which is available in the signaling pathways database. For verification of cell nucleotide sequences for cell fitness, value of biomarkers related to cell fitness is required which is available in the inflammatory and Endoplasmic Reticulum (ER) stress database. The identifying module 219 may retain the verified one or more feasible cell nucleotide sequences 115 among the plurality of cell nucleotide sequences 113 and discard the unfeasible cell nucleotide sequences among the plurality of cell nucleotide sequences 113. Consider an exemplary scenario mentioned below used to identify one or more feasible cell nucleotide sequences 115:

### Example

Query: Does the predicted cell nucleotide sequence 1 for immune evasion function predicted match with known scientific knowledge? (Query includes cell/target nucleotide sequence/biomarker data etc.) Result: “No”
Explanation: Predicted cell nucleotide sequence 1 has known adverse impact, which triggers an immune signaling pathway.

Further, the identifying module 219 may select one or more of the plurality of cell nucleotide sequences 113 within predefined threshold ranges of at least one of immune evasion and cell fitness. The predefined threshold ranges may be threshold ranges for a human body. The threshold ranges may be available in the threshold range database present in the reference information database 111.

Thereafter, the explanation generator 221 may generate the ranked list of the one or more feasible cell nucleotide sequences 115 based on an equilibrium dissociation constant (K_{D}). The one or more feasible cell nucleotide sequences 115 with lowest K_{D} value has the highest binding affinity and is given the highest ranking in the ranked list. The ranked list may be arranged as per highest to lowest ranking of the binding affinity.

In an embodiment, the explanation generator 221 may be configured for generating an explanation 117 for the ranked list of the one or more feasible cell nucleotide sequences 115, thereby designing the cell nucleotide sequences. The explanation generator 221 may utilize at least one of inverse folding, concept activation vectors and causality techniques for generating the explanation 117.

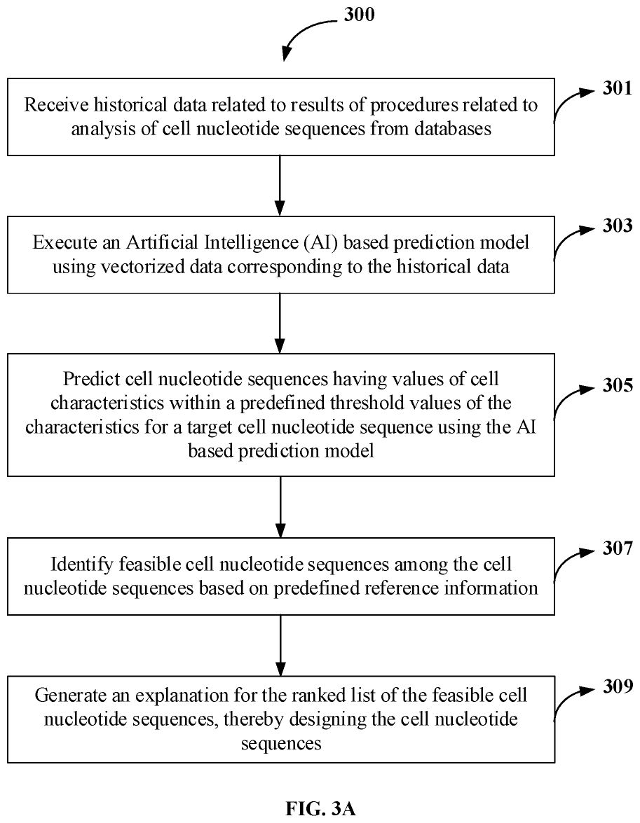

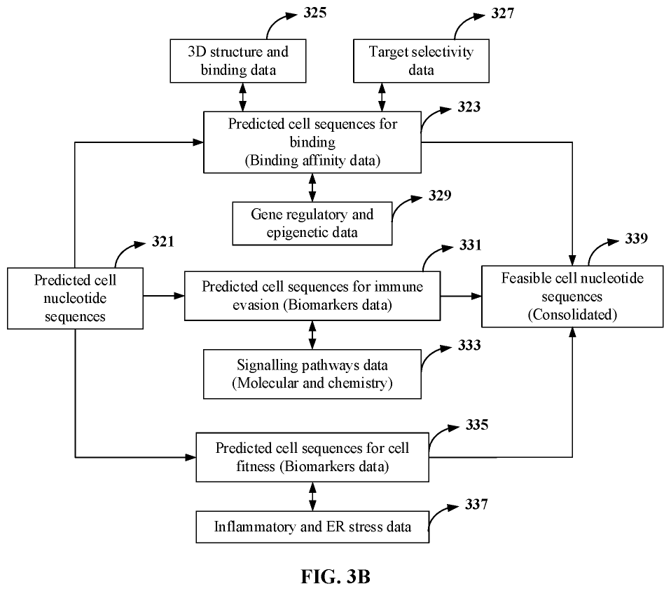

FIG. 3 shows a flowchart illustrating a method for designing cell nucleotide sequences, in accordance with some embodiments of the present disclosure.

As illustrated in FIG. 3, the method 300 may include one or more blocks illustrating a method for designing cell nucleotide sequences using a sequence designing system 101 illustrated in FIG. 2. The method 300 may be described in the general context of computer executable instructions. Generally, computer executable instructions can include routines, programs, objects, components, data structures, procedures, modules, and functions, which perform specific functions or implement specific abstract data types.

The order in which the method 300 is described is not intended to be construed as a limitation, and any number of the described method blocks can be combined in any order to implement the method. Additionally, individual blocks may be deleted from the methods without departing from the scope of the subject matter described herein. Furthermore, the method can be implemented in any suitable hardware, software, firmware, or combination thereof.

At block 301, the method 300 includes receiving, by a processor 203, historical data 103 related to results of one or more procedures related to analysis of cell nucleotide sequences from one or more databases 105. The historical data 103 may include, without limitation, at least one of one or more antigen nucleotide sequences, one or more cell nucleotide sequences, biomarkers data, function-specific biomarkers data, clinical trial progression data, clinical trial outcome data, in-vitro progression data, and in-vitro outcome data. In an embodiment, the processor 203 may perform one or more pre-processing operations on the historical data 103 for verifying correctness and completeness of the historical data 103 based on the predefined reference information 109. The predefined reference information 109 may include, without limitation, at least one of a three-dimensional (3D) structure and binding database, a gene regulatory and epigenetic database, a target selectivity database, a signaling pathways database, an inflammatory and Endoplasmic Reticulum (ER) stress database, and a threshold range database. Further, the processor 203 may arrange the historical data 103 in a chronological order based on a timestamps associated with of the historical data 103.

At block 303, the method 300 includes executing, by the processor 203, an Artificial Intelligence (AI) based prediction model 107 using vectorized data corresponding to the historical data 103.

At block 305, the method 300 includes predicting, by the processor 203, a plurality of cell nucleotide sequences 113 having values of one or more cell characteristics 211 within a predefined threshold values of the one or more cell characteristics 211 for a target cell nucleotide sequence using the AI based prediction model 107. The one or more cell characteristics comprises at least one of binding affinity, immune evasion, and cell fitness. In an embodiment, the processor 203 may determine an equilibrium dissociation constant (K_{D}) related to binding affinity, value of biomarkers related to at least one of immune evasion and cell fitness using the AI based prediction model 107.

At block 307, the method 300 includes identifying, by the processor 203, one or more feasible cell nucleotide sequences 115 among the plurality of cell nucleotide sequences 113 based on predefined reference information 109. The one or more feasible cell nucleotide sequences 115 is each assigned with a rank to generate a ranked list. In an embodiment, the processor 203 may extract a feasibility data corresponding to feasibility of each of the plurality of cell nucleotide sequences 113 from the predefined reference information 109, based on an equilibrium dissociation constant (K_{D}) and value of biomarkers related to at least one of immune evasion and cell fitness. In an embodiment, the processor 203 may select one or more of the plurality of cell nucleotide sequences 113 within predefined threshold ranges of at least one of immune evasion and cell fitness. Further, the processor 203 may generate the ranked list of the one or more feasible cell nucleotide sequences 115 based on an equilibrium dissociation constant (K_{D}).

At block 309, the method 300 includes generating, by the processor 203, an explanation 117 for the ranked list of the one or more feasible cell nucleotide sequences 115, thereby designing the cell nucleotide sequences.

FIG. 3B shows a flowchart illustrating a method for identifying one or more feasible cell nucleotide sequences 115, in accordance with some embodiments of the present disclosure.

In an embodiment, the predefined reference information 109 may be used to identify one or more feasible cell nucleotide sequences 115 among plurality of cell nucleotide sequences 113 predicted by the AI based prediction model 107. The predefined reference information 109 may be information related to scientific information from databases such as PubMed, National Institutes of Health (NIH), National Health Service (NHS), AlphaFold and the like. The predefined reference information 109 may include, without limitation, at least one of a three-dimensional (3D) structure and binding database, a gene regulatory and epigenetic database, a target selectivity database, a signaling pathways database, an inflammatory and Endoplasmic Reticulum (ER) stress database, and a threshold range database.

In an embodiment, the predefined reference information 109 may be used to verify the plurality of cell nucleotide sequences 113 (step 321) predicted by the AI based prediction model 107. For verification of cell nucleotide sequences for binding affinity (step 323) intermediate 3D structure level prediction corresponding to the cell nucleotide sequence modifications and binding affinity data (K_{D}) to the identified target is required which is available in the 3D structure and binding database (step 325), the target selectivity database (step 327) and the gene regulatory and the epigenetic database (step 329). For verification of cell nucleotide sequences for immune evasion (step 331), value of biomarkers related to immune evasion is required which is available in the signaling pathways database (step 333). For verification of cell nucleotide sequences for cell fitness (step 335), value of biomarkers related to cell fitness is required which is available in the inflammatory and Endoplasmic Reticulum (ER) stress database (step 337). The sequence designing system 101 may retain the verified one or more feasible cell nucleotide sequences 115 (step 339) among the plurality of cell nucleotide sequences 113 and discard the unfeasible cell nucleotide sequences among the plurality of cell nucleotide sequences 113.

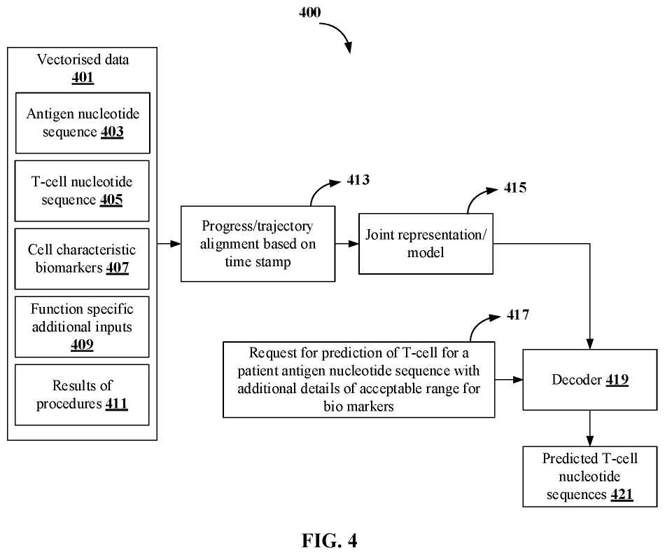

FIG. 4 shows an exemplary architecture of the proposed sequence designing system 101, in accordance with some embodiments of the present disclosure.

In an embodiment, historical data 103 may be vectorized using known vectorizing techniques. The vectorized data 401 may include, without limitation, at least one of one or more antigen nucleotide sequences 403, one or more T-cell nucleotide sequences 405, cell characteristics biomarkers data 407, function-specific additional inputs 409, and results of one or more procedures 411. Further, the vectorized data 403 may be aligned based on the timestamps (step 413) associated with the historical data 103. Upon aligning the vectorized data based on the timestamps, a joint representation/model (step 415) may be generated and used to predict a plurality of cell nucleotide sequences 113.

In an embodiment, a user may request for prediction of T-cell for a patient antigen nucleotide sequence with additional details of acceptable range for bio markers to a decoder 419 (step 417). The decoder 419 may use the joint representation/model to predict T-cell nucleotide sequences 421 based on the inputs received from the user.

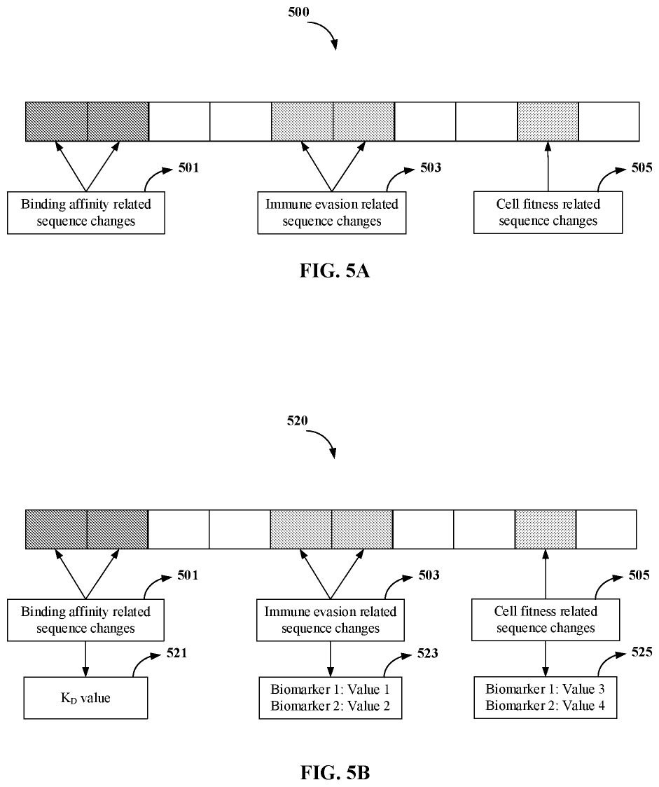

FIG. 5A shows an exemplary illustration of a predicted T-cell sequence, in accordance with some embodiments of the present disclosure.

As illustrated in FIG. 5A the predicted T-cell nucleotide sequence may indicate the binding affinity related sequence changes 501, which may be measured based on an equilibrium dissociation constant (K_{D}). Further, the predicted T-cell nucleotide sequence may indicate the immune evasion related sequence changes 503. Thereafter, the predicted T-cell nucleotide sequence may indicate the cell fitness related sequence changes 505.

FIG. 5B shows an exemplary illustration of a predicted T-cell sequence with explanation 117 and values, in accordance with some embodiments of the present disclosure.

As illustrated in FIG. 5B the predicted T-cell nucleotide sequence may indicate the binding affinity related sequence changes 501, which may be measured based on an equilibrium dissociation constant (K_{D}). The K_{D} value 521 may also be indicated to the user with the explanation 117. Further, the predicted T-cell nucleotide sequence may indicate the immune evasion related sequence changes 503. The biomarker values 523 associated immune evasion may also be indicated to the user with the explanation 117. Thereafter, the predicted T-cell nucleotide sequence may indicate the cell fitness related sequence changes 505. The biomarker values 525 associated cell fitness may also be indicated to the user with the explanation 117.

### Computer System

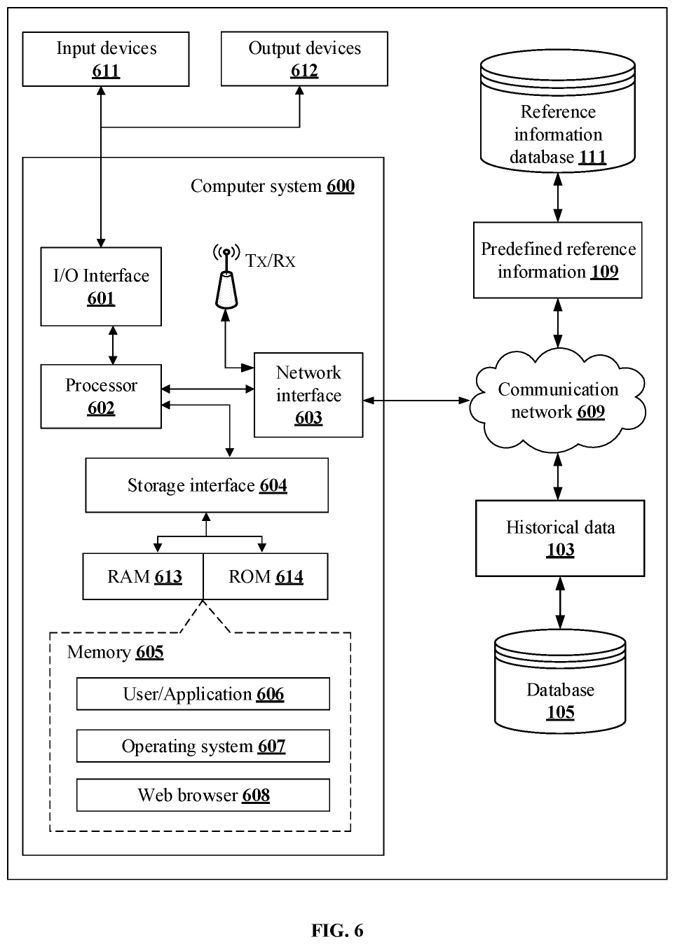

FIG. 6 illustrates an exemplary computer system 600 communicating with one or more databases (111 and 105) via communication network 609, for designing cell nucleotide sequence. In an embodiment, the computer system 600 may be the sequence designing system 101 illustrated in FIG. 2, which may be used for designing cell nucleotide sequences. The computer system 600 may include a central processing unit (“CPU” or “processor” or “memory controller”) 602. The processor 602 may comprise at least one data processor for executing program components for executing user- or system-generated business processes. A user may include a researcher, a pathologist, a qualified medical professional or any system/sub-system being operated parallelly to the computer system 600. The processor 602 may include specialized processing units such as integrated system (bus) controllers, memory controllers/memory management control units, floating point units, graphics processing units, digital signal processing units, etc.

The processor 602 may be disposed in communication with one or more Input/Output (I/O) devices (611 and 612) via I/O interface 601. The I/O interface 601 may employ communication protocols/methods such as, without limitation, audio, analog, digital, stereo, IEEER-1394, serial bus, Universal Serial Bus (USB), infrared, PS/2, BNC, coaxial, component, composite, Digital Visual Interface (DVI), high-definition multimedia interface (HDMI), Radio Frequency (RF) antennas, S-Video, Video Graphics Array (VGA), IEEER 802.n/b/g/n/x, Bluetooth, cellular (e.g., Code-Division Multiple Access (CDMA), High-Speed Packet Access (HSPA+), Global System For Mobile Communications (GSM), Long-Term Evolution (LTE) or the like), etc. Using the I/O interface 601, the computer system 600 may communicate with one or more I/O devices 611 and 612.

In some embodiments, the processor 602 may be disposed in communication with a communication network 609 via a network interface 603. The network interface 603 may communicate with the communication network 609. The network interface 603 may employ connection protocols including, without limitation, direct connect, Ethernet (e.g., twisted pair 10/100/1000 Base T), Transmission Control Protocol/Internet Protocol (TCP/IP), token ring, IEEE 802.11a/b/g/n/x, etc.

In an implementation, the communication network 609 may be implemented as one of the several types of networks, such as intranet or Local Area Network (LAN) and such within the organization. The communication network 609 may either be a dedicated network or a shared network, which represents an association of several types of networks that use a variety of protocols, for example, Hypertext Transfer Protocol (HTTP), Transmission Control Protocol/Internet Protocol (TCP/IP), Wireless Application Protocol (WAP) etc., to communicate with each other. Further, the communication network 609 may include a variety of network devices, including routers, bridges, servers, computing devices, storage devices, etc. In an embodiment, the communication network 609 may be used for interfacing with a database 105 for receiving historical data 103. Similarly, the communication network 609 may be used for interfacing with a reference information database 111 for receiving a predefined reference information 109.

In some embodiments, the processor 602 may be disposed in communication with a memory 605 (e.g., RAM 613, ROM 614, etc. as shown in FIG. 6) via a storage interface 604. The storage interface 604 may connect to memory 605 including, without limitation, memory drives, removable disc drives, etc., employing connection protocols such as Serial Advanced Technology Attachment (SATA), Integrated Drive Electronics (IDE), IEEE-1394, Universal Serial Bus (USB), fiber channel, Small Computer Systems Interface (SCSI), etc. The memory drives may further include a drum, magnetic disc drive, magneto-optical drive, optical drive, Redundant Array of Independent Discs (RAID), solid-state memory devices, solid-state drives, etc.

The memory 605 may store a collection of program or database components, including, without limitation, user/application interface 606, an operating system 607, a web browser 608, and the like. In some embodiments, computer system 600 may store user/application data 606, such as the data, variables, records, etc. as described in this invention. Such databases may be implemented as fault-tolerant, relational, scalable, secure databases such as Oracle® or Sybase®.

The operating system 607 may facilitate resource management and operation of the computer system 600. Examples of operating systems include, without limitation, APPLE® MACINTOSH® OS X®, UNIX®, UNIX-like system distributions (E.G., BERKELEY SOFTWARE DISTRIBUTION® (BSD), FREEBSD®, NETBSD®, OPENBSD, etc.), LINUX® DISTRIBUTIONS (E.G., RED HAT®, UBUNTU®, KUBUNTU®, etc.), IBM® OS/2®, MICROSOFT® WINDOWS® (XP®, VISTA®/7/8, 10 etc.), APPLE® IOS®, GOOGLE™ ANDROID™, BLACKBERRY® OS, or the like.

The user interface 606 may facilitate display, execution, interaction, manipulation, or operation of program components through textual or graphical facilities. For example, the user interface 606 may provide computer interaction interface elements on a display system operatively connected to the computer system 600, such as cursors, icons, check boxes, menus, scrollers, windows, widgets, and the like. Further, Graphical User Interfaces (GUIs) may be employed, including, without limitation, APPLE® MACINTOSH® operating systems' Aqua®, IBM® OS/2®, MICROSOFT® WINDOWS® (e.g., Aero, Metro, etc.), web interface libraries (e.g., ActiveX®, JAVA®, JAVASCRIPT®, AJAX, HTML, ADOBE® FLASH®, etc.), or the like.

The web browser 608 may be a hypertext viewing application. Secure web browsing may be provided using Secure Hypertext Transport Protocol (HTTPS), Secure Sockets Layer (SSL), Transport Layer Security (TLS), and the like. The web browsers 608 may utilize facilities such as AJAX, DHTML, ADOBE® FLASH®, JAVASCRIPT®, JAVA®, Application Programming Interfaces (APIs), and the like. Further, the computer system 600 may implement a mail server stored program component. The mail server may utilize facilities such as ASP, ACTIVEX®, ANSI® C++/C#, MICROSOFT®, .NET, CGI SCRIPTS, JAVA®, JAVASCRIPT®, PERL®, PHP, PYTHON®, WEBOBJECTS®, etc. The mail server may utilize communication protocols such as Internet Message Access Protocol (IMAP), Messaging Application Programming Interface (MAPI), MICROSOFT® exchange, Post Office Protocol (POP), Simple Mail Transfer Protocol (SMTP), or the like. In some embodiments, the computer system 600 may implement a mail client stored program component. The mail client may be a mail viewing application, such as APPLE® MAIL, MICROSOFT® ENTOURAGE®, MICROSOFT® OUTLOOK®, MOZILLA® THUNDERBIRD®, and the like.

Furthermore, one or more computer-readable storage media may be utilized in implementing embodiments consistent with the present invention. A computer-readable storage medium refers to any type of physical memory on which information or data readable by a processor may be stored. Thus, a computer-readable storage medium may store instructions for execution by one or more processors, including instructions for causing the processor(s) to perform steps or stages consistent with the embodiments described herein. The term “computer-readable medium” should be understood to include tangible items and exclude carrier waves and transient signals, i.e., non-transitory. Examples include Random Access Memory (RAM), Read-Only Memory (ROM), volatile memory, nonvolatile memory, hard drives, Compact Disc (CD) ROMS, Digital Video Disc (DVDs), flash drives, disks, and any other known physical storage media.

### Advantages of the Embodiments of the Present Disclosure are Illustrated Herein

In an embodiment, the present disclosure provides an Artificial Intelligence (AI) based prediction model to design cell nucleotide sequences that do not generate adverse immune responses, demonstrate cell fitness and display substantial binding capacity against the target antigens.

In an embodiment, the present disclosure helps in predicting and identifying one or more feasible cell nucleotide sequences among a large number of possible cell nucleotide sequences. Consequently, the present disclosure helps to design successful cell nucleotide sequences at a faster rate with high efficiency as the number of possible sequences has been reduced.

In an embodiment, the present disclosure generates an explanation for the one or more feasible nucleotide sequences. This helps a user, such as a doctor, to understand the values of the one or more cell characteristics of the identified one or more feasible nucleotide sequences.

As stated above, it shall be noted that the method of the present disclosure may be used to overcome various technical problems related to designing cell nucleotide sequence. In other words, the disclosed method has a practical application and provides a technically advanced solution to the technical problems associated with the existing sequence designing system.

In light of the technical advancements provided by the disclosed method and the sequence designing system, the claimed steps, as discussed above, are not routine, conventional, or well-known aspects in the art, as the claimed steps provide the aforesaid solutions to the technical problems existing in the conventional technologies. Further, the claimed steps clearly bring an improvement in the functioning of the system itself, as the claimed steps provide a technical solution to a technical problem.

The terms “an embodiment”, “embodiment”, “embodiments”, “the embodiment”, “the embodiments”, “one or more embodiments”, “some embodiments”, and “one embodiment” mean “one or more (but not all) embodiments of the invention(s)” unless expressly specified otherwise.

The terms “including”, “comprising”, “having” and variations thereof mean “including but not limited to”, unless expressly specified otherwise.

The enumerated listing of items does not imply that any or all the items are mutually exclusive, unless expressly specified otherwise. The terms “a”, “an” and “the” mean “one or more”, unless expressly specified otherwise.

A description of an embodiment with several components in communication with each other does not imply that all such components are required. On the contrary, a variety of optional components are described to illustrate the wide variety of possible embodiments of the invention.

When a single device or article is described herein, it will be clear that more than one device/article (whether they cooperate) may be used in place of a single device/article. Similarly, where more than one device/article is described herein (whether they cooperate), it will be clear that a single device/article may be used in place of the more than one device/article or a different number of devices/articles may be used instead of the shown number of devices or programs. The functionality and/or features of a device may be alternatively embodied by one or more other devices which are not explicitly described as having such functionality/features. Thus, other embodiments of invention need not include the device itself.

Finally, the language used in the specification has been principally selected for readability and instructional purposes, and it may not have been selected to delineate or circumscribe the inventive subject matter. It is therefore intended that the scope of the invention be limited not by this detailed description, but rather by any claims that issue on an application based here on. Accordingly, the embodiments of the present invention are intended to be illustrative, but not limiting, of the scope of the invention, which is set forth in the following claims.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

### REFERRAL NUMERALS

| Reference Number | Description |
|---|---|
| 101 | Sequence designing system |
| 103 | Historical data |
| 105 | Databases |
| 107 | Prediction model |
| 109 | Predefined reference information |
| 111 | Reference information database |
| 113 | Plurality of cell nucleotide sequences |
| 115 | Feasible cell nucleotide sequences |
| 117 | Explanation |
| 201 | I/O Interface |
| 203 | Processor |
| 205 | Memory |
| 207 | Data |
| 209 | Modules |
| 211 | Cell characteristics |
| 213 | Other data |
| 215 | Receiving module |
| 217 | Executing module |
| 219 | Identifying module |
| 221 | Explanation generator |
| 223 | Other modules |
| 600 | Exemplary computer system |
| 601 | I/O Interface of the exemplary computer system |
| 602 | Processor of the exemplary computer system |
| 603 | Network interface |
| 604 | Storage interface |
| 605 | Memory of the exemplary computer system |
| 606 | User/Application |
| 607 | Operating system |
| 608 | Web browser |
| 609 | Communication network |
| 611 | Input devices |
| 612 | Output devices |
| 613 | RAM |
| 614 | ROM |

What is claimed is:

1. A computer implemented method of designing cell nucleotide sequences, the computer implemented method comprising:
receiving, by a sequence designing system, historical data related to results of one or more procedures related to analysis of cell nucleotide sequences from one or more databases;
executing, by the sequence designing system, an Artificial Intelligence (AI) based prediction model using vectorized data corresponding to the historical data;
predicting, by the sequence designing system, a plurality of cell nucleotide sequences having values of one or more cell characteristics within a predefined threshold values of the one or more cell characteristics for a target cell nucleotide sequence using the AI based prediction model;
identifying, by the sequence designing system, one or more feasible cell nucleotide sequences among the plurality of cell nucleotide sequences based on predefined reference information, wherein the one or more feasible cell nucleotide sequences is each assigned with a rank to generate a ranked list; and
generating, by the sequence designing system, an explanation for the ranked list of the one or more feasible cell nucleotide sequences, thereby designing the cell nucleotide sequences.

2. The computer implemented method as claimed in claim 1, wherein receiving the historical data further comprises:
performing, by the sequence designing system, one or more pre-processing operations on the historical data for verifying correctness and completeness of the historical data based on the predefined reference information; and
arranging, by the sequence designing system, the historical data in a chronological order based on timestamps associated with the historical data.

3. The computer implemented method as claimed in claim 1, wherein predicting the plurality of cell nucleotide sequences comprises:
determining, by the sequence designing system, an equilibrium dissociation constant (K_{D}) related to binding affinity, value of biomarkers related to at least one of immune evasion and cell fitness using the AI based prediction model.

4. The computer implemented method as claimed in claim 1, wherein identifying the one or more feasible cell nucleotide sequences comprises:
extracting, by the sequence designing system, a feasibility data corresponding to feasibility of each of the plurality of cell nucleotide sequences from the predefined reference information, based on an equilibrium dissociation constant (K_{D}) and value of biomarkers related to at least one of immune evasion and cell fitness.

5. The computer implemented method as claimed in claim 1, wherein identifying the one or more feasible cell nucleotide sequences further comprises:
selecting, by the sequence designing system, one or more of the plurality of cell nucleotide sequences within predefined threshold ranges of at least one of immune evasion and cell fitness; and
generating, by the sequence designing system, the ranked list of the one or more feasible cell nucleotide sequences based on an equilibrium dissociation constant (K_{D}).

6. The computer implemented method as claimed in claim 1, wherein the historical data comprises at least one of one or more antigen nucleotide sequences, one or more cell nucleotide sequences, biomarkers data, function-specific biomarkers data, clinical trial progression data, clinical trial outcome data, in-vitro progression data, and in-vitro outcome data.

7. The computer implemented method as claimed in claim 1, wherein the one or more cell characteristics comprises at least one of binding affinity, immune evasion, and cell fitness.

8. The computer implemented method as claimed in claim 1, wherein the predefined reference information comprises at least one of a three-dimensional (3D) structure and binding database, a gene regulatory and epigenetic database, a target selectivity database, a signaling pathways database, an inflammatory and Endoplasmic Reticulum (ER) stress database, and a threshold range database.

9. A sequence designing system for designing cell nucleotide sequence, the sequence designing system comprising:
a processor; and
a memory, communicatively coupled to the processor, wherein the memory stores processor-executable instructions, which, on execution, causes the processor to:
receive historical data related to results of one or more procedures related to analysis of cell nucleotide sequences from one or more databases;
execute an Artificial Intelligence (AI) based prediction model using vectorized data corresponding to the historical data;
predict a plurality of cell nucleotide sequences having values of one or more cell characteristics within a predefined threshold values of the one or more cell characteristics for a target cell nucleotide sequence using the AI based prediction model;
identify one or more feasible cell nucleotide sequences among the plurality of cell nucleotide sequences based on predefined reference information, wherein the one or more feasible cell nucleotide sequences is each assigned with a rank to generate a ranked list; and
generate an explanation for the ranked list of the one or more feasible cell nucleotide sequences, thereby designing the cell nucleotide sequences.

10. The sequence designing system as claimed in claim 9, wherein after receiving the historical data, the processor is further configured to:
perform one or more pre-processing operations on the historical data for verifying correctness and completeness of the historical data based on the predefined reference information; and
arrange the historical data in a chronological order based on timestamps associated with the historical data.

11. The sequence designing system as claimed in claim 9, wherein the processor predicts the plurality of cell nucleotide sequences by:
determining an equilibrium dissociation constant (K_{D}) related to binding affinity, value of biomarkers related to at least one of immune evasion and cell fitness using the AI based prediction model.

12. The sequence designing system as claimed in claim 9, wherein the processor identifies the one or more feasible cell nucleotide sequences by:
extracting a feasibility data corresponding to feasibility of each of the plurality of cell nucleotide sequences from the predefined reference information, based on an equilibrium dissociation constant (K_{D}) and value of biomarkers related to at least one of immune evasion and cell fitness.

13. The sequence designing system as claimed in claim 9, wherein after identifying the one or more feasible cell nucleotide sequences, the processor is further configured to:
select one or more of the plurality of cell nucleotide sequences within predefined threshold ranges of at least one of immune evasion and cell fitness; and
generate the ranked list of the one or more feasible cell nucleotide sequences based on an equilibrium dissociation constant (K_{D}).

14. The sequence designing system as claimed in claim 9, wherein the historical data comprises at least one of one or more antigen nucleotide sequences, one or more cell nucleotide sequences, biomarkers data, function-specific biomarkers data, clinical trial progression data, clinical trial outcome data, in-vitro progression data, and in-vitro outcome data.

15. The sequence designing system as claimed in claim 9, wherein the one or more cell characteristics comprises at least one of binding affinity, immune evasion, and cell fitness.

16. The sequence designing system as claimed in claim 9, wherein the predefined reference information comprises at least one of a three-dimensional (3D) structure and binding database, a gene regulatory and epigenetic database, a target selectivity database, a signaling pathways database, an inflammatory and Endoplasmic Reticulum (ER) stress database, and a threshold range database.

17. A non-transitory computer readable medium including instructions stored thereon that when processed by at least one processor, cause a sequence designing system to perform operations comprising:
receiving historical data related to results of one or more procedures related to analysis of cell nucleotide sequences from one or more databases;
executing an Artificial Intelligence (AI) based prediction model using vectorized data corresponding to the historical data;
predicting a plurality of cell nucleotide sequences having values of one or more cell characteristics within a predefined threshold values of the one or more cell characteristics for a target cell nucleotide sequence using the AI based prediction model;
identifying one or more feasible cell nucleotide sequences among the plurality of cell nucleotide sequences based on predefined reference information, wherein the one or more feasible cell nucleotide sequences is each assigned with a rank to generate a ranked list; and
generating an explanation for the ranked list of the one or more feasible cell nucleotide sequences, thereby designing the cell nucleotide sequences.
